(19) United States
(12) Patent Application Publication
(10) Pub. No.: US 2026/0199632 A1
(43) Pub. Date: Jul. 16, 2026

(54) CHROMIC SENSOR FOR AN INTRAVENOUS CATHETER

(71) Applicant: CareFusion 303, Inc., San Diego, CA (US)

(72) Inventors: Sara RUTH, Englewood, NJ (US); Cherry VANOVER, Draper, UT (US); Andrew Latimer RICHARDS, Durham, NC (US); Leandro FORCINITI, Haskell, NJ (US); Shishir PRASAD, Ramsey, NJ (US); Asli Zeynep UNAL, Lincolnshire, IL (US); Eugene E. MASON, La Habra Heights, CA (US); Joyce L. YOUNG-STEWART, Sparks, NV (US); Christopher James RINI, Raleigh, NC (US)

(21) Appl. No.: 19/022,916

(22) Filed: Jan. 15, 2025

(51) Int. Cl. A61M 25/00; A61B 5/145; A61B 5/1455

(52) U.S. Cl. CPC A61M 25/0068; A61B 5/14539; A61B 5/1455; A61M 2205/3306; A61M 2205/6081; A61M 2205/702

(57) ABSTRACT

A chromic sensor for an intravenous catheter may include a first region disposed to be proximal to an insertion site at which the intravenous catheter is inserted into a patient. The first region includes a chromic material that changes color in response to a stimulus. A second region of the chromic sensor is disposed to be distal to the insertion site, spaced apart from the first region along a portion of the intravenous catheter inserted into the patient. The second region includes the chromic material. The first and second regions are configured to respond identically to identical stimuli.

### TECHNICAL FIELD

The present disclosure generally relates to intravenous catheters, in particular, to devices and methods used for monitoring a catheter insertion site.

### BACKGROUND

Catheter site assessments are routine observations performed by nurses (or physicians, technicians, and other clinicians) on patients that have an intravenous catheter (peripheral or central). The nurse inspects the catheter insertion site to confirm whether: the catheter is patent and functioning; the catheter is properly in place and has not been dislodged; the dressing is secure, is not peeling, dirty or otherwise compromised; and the skin around the catheter insertion site is not discolored, warm, or soft.

Site assessments can provide early detection and intervention if there are any catheter-related complications (including infiltrations, extravasations, phlebitis, dislodgement, etc.) or local site infections. These assessments are typically performed periodically, depending on the type of catheter, infusate, and patient-specific risk factors. For example, infusing certain medications such as vancomycin, which may be venous irritants through a peripheral IV catheter may require a nurse to assess the catheter site every two hours to ensure there is no infiltration. In some care settings such a critical care and oncology, the evaluation frequency can be more frequent. The site assessments can be performed as a standalone observation or as part of another procedure (medication delivery, dressing change, overall patient assessments etc.). The site assessments are documented in the electronic medical record (EMR).

Site assessments typically involve the following: inspecting the IV catheter insertion site for redness, swelling, or bruising (the patient's other extremity may be utilized for comparison); assessing for tenderness and swelling through palpating the skin around the catheter site; assessing the condition of the transparent catheter dressing, including if it is clean, dry, and securely adhered to the skin around the insertion site; assessing if any additional securement is used to keep the dressing intact (e.g. additional tape, bandages etc.); assessing the catheter for patency, typically by flushing the catheter with saline or heparin; and assessing products used to secure and stabilize the catheter (particularly for central lines). In the EMR, the nurse will also document the reasons for continuing to use the catheter, and assess for signs of infiltration, phlebitis, dislodgement, and migration.

There are several challenges with site assessments. For example, site assessments are inherently subjective, being heavily dependent on the observer's awareness and experience; a less experienced floor nurse may observe a site to appear normal while a trained nurse may spot early signs of infiltration or phlebitis; patient-specific factors (e.g. skin color, skin folds, natural discoloration) can confound results; variability in products used to secure and stabilize catheters, leading to variable catheter-related outcomes; little data or evidence to help nurses identify what is an acceptable versus unacceptable catheter site (e.g. how much dressing peeling is acceptable, how much local redness is acceptable); due to documentation overload, nurses may copy and paste assessments, without even observing it on the patient; high frequency site assessments create a workflow and documentation burden for the nurse, which increases likelihood of resorting to short-cuts; site-assessments may not be conducted in a timely manner which would otherwise allow for early and timely indication of a complication, such as phlebitis; and site-assessments are not tracked or validated for accuracy since the only data available is that documented by the nurse in the EMR. There is also an opportunity to evaluate if site assessments can be used as predictors of an impending catheter-related complication, including comparison of a site over time to enable earlier detecting of complications.

In some instances of a catheter site complication, such as phlebitis, the patient may have an inflammatory reaction at or around the catheter insertion site. The body of the patient may have an inflammatory reaction to the catheter which can raise the local temperature around the catheter insertion site at which the catheter enters the skin. There can be many other sources of local temperature difference related to catheters, such as reactions to drugs introduced by the catheter, fluids leaked outside the vein (e.g. infiltration, extravasation, etc.), reactions to pistoning or mechanical catheter motion in the body, immune responses to infectious agents such as bacteria and biofilms that form on the catheter surface or inside the lumen, the temperature of the IV fluid relative to the body or ambient temperature, foreign body reactions to the catheter materials and surface, and/or the like.

Further, increased temperature at a catheter insertion site may be due to both infection and phlebitis or other irritation to the vein caused by the catheter or a fluid running through the catheter. Additionally, decreases in temperature at a catheter insertion site may also indicate a problem because these decreases in temperature may be caused when fluid leaks into the tissue from the infusion. This leaked fluid can lead to outcomes as minor as pain or, in some cases, tissue damage resulting in loss of a limb. Moreover, the catheter tubing and the fluid running therethrough may provide a source of temperature variation because the fluid being delivered is often at room temperature or chilled as opposed to being at a body temperature of the patient. Accordingly, there is a need in the art for improved temperature sensing for intravenous site condition detection.

### SUMMARY

The techniques disclosed herein advantageously can provide improved site assessments at catheter insertion sites. Moreover, the techniques herein can eliminate the subjectivity in site assessments by nurses or practitioners, and provide a visual method for determining whether a catheter insertion site has been compromised. Advantageously, the result of the visual determination can be easily recorded in the electronic medical record, thereby reducing the documentation burden on the nurses or practitioner, and eliminating corresponding errors. Further advantageously, the devices disclosed herein can be incorporated in a dressing, as an attachment to an intravenous catheter, or as part of a kit including the dressing and/or the catheter. In other words, the techniques and devices provided herein do not require substantial changes in pre-existing protocols.

In addition, the use of different types of sensors/dyes in the techniques can provide for detection of different types of catheter-related complications such as, for example, local temperature changes or local immune reactions caused by potential infections, physical damage to the catheter, phlebitis, dislodgement or other forms of insertion site compromise. Even though specific use examples include the medical industry, these solutions have wide application potentials in other industries.

In one embodiment, the technology provides a chromic sensor for monitoring an insertion site at which an intravenous catheter (IVC) is inserted. The chromic sensor may include a first region and a second region The first region is disposed to be proximal to the insertion site at which the IVC is inserted into a patient. The first region includes a chromic material that changes color in response to a stimulus. The second region is disposed to be distal to the insertion site and spaced apart from the first region along a portion of the IVC inserted into the patient. The first and second regions are configured to respond identically to identical stimuli. The stimulus may include, for example, change in temperature, change in pH, mechanical stress or strain, presence of a chemical entity, or a combination thereof.

In another embodiment, the technology provides a dressing for attaching to a portion of the skin of the patient surrounding the insertion site at which the IVC is inserted into the patient. The dressing may include the chromic sensor disclosed herein. The dressing may further include a visual marker positioned to align with the insertion site when the dressing is attached to the insertion site.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 schematically shows an intravenous catheter inserted in the arm of a patient, and a chromic sensor attached to the arm of the patient adjacent the intravenous catheter in accordance with at least some embodiments of the present disclosure.

FIG. 2 shows a schematic of an intravenous catheter.

FIG. 3 shows a schematic of a chromic sensor in accordance with at least some embodiments of the present disclosure.

FIG. 4 shows a schematic of a chromic sensor including a clip in accordance with at least some embodiments of the present disclosure.

FIG. 5 shows a schematic of a dressing including a chromic sensor in accordance with at least some embodiments of the present disclosure.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

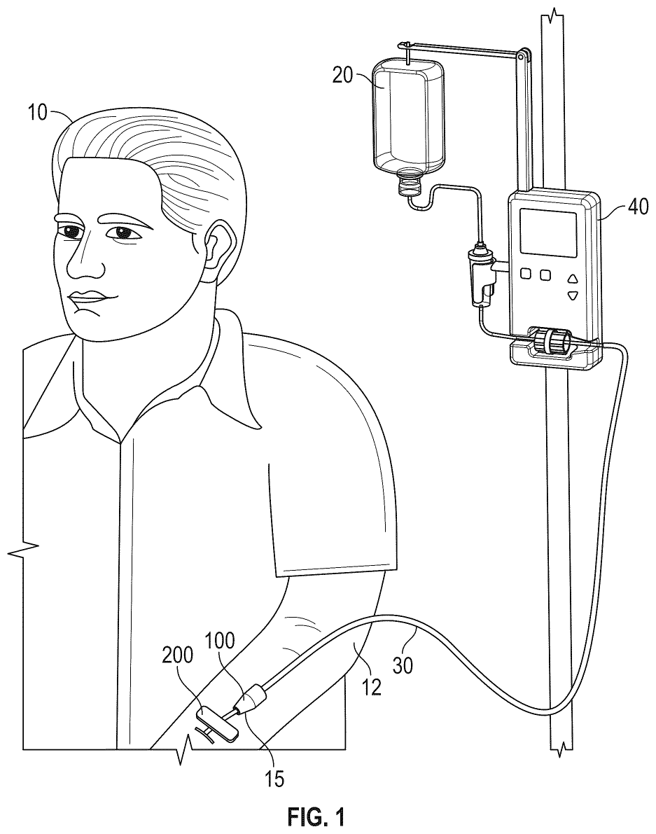

FIG. 1 schematically illustrates an intravenous catheter inserted in an arm of a patient. In use, the intravenous catheter 100 is inserted in the arm 12 of the patient 10, to facilitate delivery of fluids to the patient 10 from an IV bag 20 via medical tubing 30. In some instances, a flow regulator 40 may regulate the volume and/or rate of flow of the fluids flowing from the IV bag 20 to the patient. A chromic sensor 200 may be placed on the arm of the patient adjacent an insertion site 15 at which the intravenous catheter 100 is inserted into the arm 12 of the patient 10. It will be understood that while FIG. 1 illustrates the intravenous catheter being inserted into an arm of the patient, the catheter may be inserted into any other part of the patient's body such as, for example, the leg or torso of the patient.

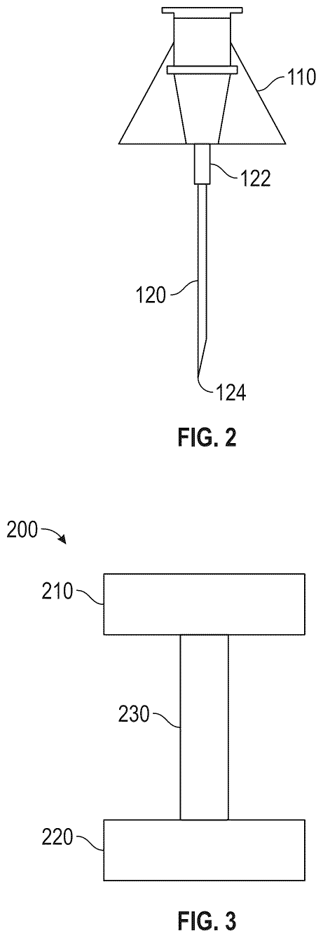

FIG. 2 illustrates a schematic of an intravenous catheter (IVC). The IVC 100 includes hub 110, and a tube 120. In use, the distal end 124 of the tube 120 is inserted through an insertion site on a portion of skin of a patient adjacent to a vessel in which the IVC is to be inserted, while the proximal end 122 remains on the surface of the skin along with the hub 110. The hub 110 may be connected to another tube (not shown) through which a medication or other liquids may be delivered to the patient via the distal end 124 of the IVC 100.

As discussed elsewhere herein, after the insertion of the IVC 100 the insertion site is monitored periodically to check, for example, that the catheter is patent and functioning; the catheter is properly in place and has not been dislodged; the dressing is secure, is not peeling, dirty or otherwise compromised; and the skin around the catheter insertion site is not discolored, warm, or soft, at the insertion site. The chromic sensor disclosed herein provides a visual aid for such monitoring.

FIG. 3 illustrates a chromic sensor in accordance with at least some embodiments of the present disclosure. In one embodiment, a chromic sensor 200 for an intravenous catheter (IVC) such as, for example, the IVC 100, includes a first region 210, a second region 220 spaced apart from the first region 210 with a connector 230 disposed therebetween.

The first region 210 is arranged to be disposed proximal to the insertion site through which the IVC is inserted into the patient while the second region 220 is arranged to be disposed distal to the insertion site along a portion of the IVC that is inserted into the patient. In other words, the first and second regions are arranged to at least partially overlap with a portion of the tube of the IVC that resides in the patient during use such that the first region is disposed proximal to the position at which the tube of the IVC is inserted into the patient, and the second region is disposed further along the length of the tube distal to the insertion site (and proximal to the distal end of the tube).

While the schematic shown in FIG. 3 illustrates a rectangular shape for the first and second regions, any suitable shape for the first and second regions is contemplated within the scope of the present disclosure. For example, the first and second regions may be circular, elliptical, polygonal, or any other suitable shape so long as the shape and size of chromic sensor can be suitably attached to the skin of the patient near an insertion site at which an IVC is inserted into the patient. Similarly, the distance between the first and second regions 210/220, i.e., the length of the connector 230, is not particularly limited so long as the distance can suitably allow for detection of a differential in stimulus between the first and second regions. Thus, the length of the connector 230 may range from about 0.5 cm to about 10 cm, depending on the length of the portion of the IVC inserted into the patient.

The first and second regions include a chromic material selected to change color in response to a stimulus. As used herein, the term “chromic material” refers to molecules, dyes, and pigments that exhibit a measurable color change in response to an external stimulus. The color change may or may not be reversible. The first and second regions include the chromic material such that both the first and second regions respond identically to identical stimuli. Thus, in use, the chromic sensor 200 provides a visual indication when there is a difference in local conditions on or underlying the skin of the patient between the first and second regions. A difference in local conditions may be indicative of a complication near the IVC insertion site. The complication may be caused, for example, by an infection, dislodging of the catheter, blockage of the catheter, leak in the catheter tube, or a change in skin condition near the insertion site, resulting in a difference in stimuli received at the first and second regions of the chromic sensor.

Without wishing to be bound by theory, the identical responses to identical stimuli at the first and second regions provide for normalization of the measurement provided by the chromic sensor 200 by eliminating environmental impact on the local responses. For example, For example, if the patient has a limb with IVC inserted therein placed inside a blanket, the temperature for both the first and second regions would change identically and the color of both sensors would appear to be same. In contrast, different responses from the two regions would indicate different local temperatures of underlying the two regions, which would give a visual feedback to the care provider that there may be a complication near the IVC insertion site.

The chromic material provided in the chromic sensor may respond to various types of stimuli such as, for example, change in temperature, change in pH, mechanical stress or strain, presence of a chemical entity, or a combination thereof. Thus, in some implementations of the chromic sensor, if the temperature of the skin underlying the first region 210 is different from the temperature of the skin underlying the second region 220, the first and second regions will respond differently, i.e., changing colors differently. On the other hand, if the temperature of the skin underlying the first and second regions is the same, the first and second regions respond identically and appear to have the same color.

In some implementations, the chromic material may be a temperature sensitive dye. Examples of temperature sensitive dyes include, but are not limited to, thermochromic liquid crystals (TLCs) or leuco dyes. TLCs can be highly temperature sensitive, and can exhibit a range of colors depending on the change in temperature. Some TLCs can reversibly change color with a change in temperature as low as 0.1° C. While TLCs can be formulated to respond to temperature changes ranging from about −30° C. to about 120° C., in implementations of the present disclosure, TLCs can be selected to be active in a temperature range from about 25° C. (room temperature, at which fluids are typically infused through the catheter) to about 40° C. (which is a typical upper limit of local skin temperature caused by an inflammation or an immune response to an infection), which is a typical range of temperatures experienced by an IVC catheter. In some implementations, the TLCs can be selected to respond to any range within the temperature range from about 25° C. to about 40° C. For example, the TLC may be selected to be response to a temperature change in a range from about 0.1° C. to about 10° C., about 0.1° C. to about 0.5° C., about 0.1° C. to about 1° C., about 0.1° C. to about 2° C., about 0.1° C. to about 3° C., about 0.1° C. to about 4° C., about 0.1° C. to about 5° C., about 0.1° C. to about 6° C., about 0.1° C. to about 7° C., about 0.1° C. to about 8° C., about 0.1° C. to about 9° C., about 0.1° C. to about 10° C., about 0.5° C. to about 5° C., about 0.5° C. to about 10° C., about 1° C. to about 5° C., about 1° C. to about 10° C., or any other range of temperature change, within a temperature range from about 25° C. to about 40° C..

Leuco dyes, on the other hand, are less sensitive compared to TLCs but also less expensive. Depending on the particular type of leuco dye, a minimum temperature change needed to cause of color change in a leuco dye may be about 3° C.. As with TLCs, leuco dyes can also be formulated to respond to temperature changes ranging from about −30° C. to about 120° C., and in implementations of the present disclosure, can be selected to be active in a temperature range from about 25° C. to about 40° C. or any temperature range therewithin. For example, the TLC may be selected to be response to a temperature change in a range from about 2.0° C. to about 10° C. within a temperature range from about 25° C. to about 40° C.. For example, the leuco dye may be selected to be response to a temperature change in a range from about 2° C. to about 10° C., about 2.0° C. to about 2.5° C., about 2° C. to about 3.0° C., about 2.0° C. to about 3.5° C., about 2.0° C. to about 4.0° C., about 2.0° C. to about 4.5° C., about 2.0° C. to about 5.0° C., about 2.0° C. to about 5.5° C., about 2.0° C. to about 6.0° C., about 2.0° C. to about 6.5° C., about 2.0° C. to about 7.0° C., about 2.0° C. to about 7.5° C., about 2.0° C. to about 8.0° C., about 2.0° C. to about 9.0° C., about 2.0° C. to about 10.0° C., about 2.5° C. to about 3.0° C., about 2.5° C. to about 4.0° C., about 2.5° C. to about 5.0° C., about 2.5° C. to about 6.0° C., about 2.5° C. to about 7.0° C., about 2.5° C. to about 8.0° C., about 2.5° C. to about 9.0° C., about 2.5° C. to about 10.0° C., about 3.0° C. to about 4.0° C., about 3.0° C. to about 5.0° C., about 3.0° C. to about 6.0° C., about 3.0° C. to about 7.0° C., about 3.0° C. to about 8.0° C., about 3.0° C. to about 9.0° C., about 3.0° C. to about 10.0° C., or any other range of temperature change, within a temperature range from about 25° C. to about 40° C..

In some implementations, the chromic sensor, and in particular, the first and second regions, may include microparticles encapsulating, embedding, or coated with one or more suitable leuco dyes or TLCs. The microparticles may be incorporated in a matrix or a capsule. In some implementations, the matrix or the capsule has a base color selected to provide high contrast relative to the color of the TLC or the leuco dye in native and/or changed state. For example, the matrix or the capsule may have a black base color. In one example, a black fabric matrix is shaped to form the first region 210, the second region 220, and the connector 230 such the microparticles are incorporated in the fabric matrix only in the first and second regions.

In some implementations, the chromic material may be a pH sensitive dye. Such a chromic material may respond to a local change in pH caused, for example, by an infection of the skin. Typical pH of healthy skin is in a range from 4.5 to 5.75. In contrast, infected skin tends to have alkaline pH ranging from about 7.2 to about 7.5. Thus, a chromic sensor including a pH sensitive chromic material may be useful in detecting an acute wound or a skin infection.

Examples of pH sensitive dyes include, but are not limited to, a sulfophthalein dye, a phthalein dye, an azo dye, a 1, 8-naphthalimide dye, a boron-dipyrromethene dye, a diketopyrrolopyrrole dye, a cyanine dye, or a fluorescein. The pH sensitive dyes may be incorporated in a matrix or a fabric to form the chromic sensor in a suitable quantity to allow detection of the pH change in a range from about 0.5 to about 3.5, about 1.0 to about 3.0, about 1.0 to about 2.5, about 1.5 to about 3.0, about 1.5 to about 2.5, or any other range of pH change therebetween.

In some implementations, the chromic material may be a mechanochromic dye that changes color in response to mechanical strain, for example, caused by an inflammation of the skin (e.g., swelling), dislodging of the catheter from its intended position, leakage from the catheter, or other causes that may lead to mechanical stress or distortion around the chromic sensor. The mechanical strain caused by such changes to local skin conditions may be in a range from 0.5% to about 3.0%, for example, from about 0.5% to about 1.0%, about 0.5% to about 1.5%, about 0.5% to about 2.0%, about 1.0% to about 1.5%, about 1.0% to about 2.0%, 1.5% to about 2.0%, about 1.5% to about 2.5%, about 1.5% to about 3.0%, about 2.0% to about 3.0%, or any range of strain therebetween.

Examples of mechanochromic dyes include, but are not limited to, spiropyrans, spirooxazines, diarylethenes, metal-organic frameworks, liquid crystal polymers or a combination thereof. The mechanochromic dyes may be incorporated in a matrix or a fabric to form the chromic sensor. Alternately, the chromic sensor include microparticles encapsulating, embedding, or coated with one or more suitable mechanochromic dyes. The microparticles may be incorporated in a matrix or a capsule. In some implementations, the matrix or the capsule has a base color selected to provide high contrast relative to the color of the mechanochromic dye in native and/or changed state.

In some implementations, the chromic material may be a solvent reactive dye which changes color in response to contact with extracellular fluids, for example, when the extracellular fluids leak out of the skin surrounding the insertion site. The extracellular fluids may leak out of the skin because of a puncture near the insertion site. The puncture may be caused by, for example, the catheter dislodging or an injury to the skin near the catheter insertion site. The solvent reactive dye may, for example, be sensitive to particular salts (or concentrations thereof) or antigens present in the extracellular fluids by producing a color change response. In some instances, the solvent reactive dye may be a pH sensitive dye discussed elsewhere herein. The solvent sensitive dye may be incorporated into a fabric or a matrix that is disposed such that when in contact with the extracellular fluid, the dye disposed therein responds to the presence of the extracellular fluid by changing color.

In some implementations, the chromic material may be a sensitive to a particular biomarker such as, for example, a particular antibody or toxin. For example, the biomarker may be a toxin indicative of an infection, or a toxin released by a bacteria or yeast. Additionally or alternatively, the biomarker may be an immunoglobulin or a cytokine that is released in response to an infection or inflammation.

The chromic material in such implementations may be formed by incorporating an antibody tagged with chromic marker such as a fluorescent marker or a dye in a suitable polymer matrix such as, for example, polyacrylic acid, hyaluronic acid, polyethylene glycol, or a combination thereof. Other suitable biocompatible and/or biodegradable polymers are contemplated within the scope of the present disclosure. The polymer matrix may be formulated into microparticles which can be coated onto to a fabric or other matrix to form the first and second regions of the chromic sensor. In some implementations, the polymer matrix may be formed into the first and second regions of the chromic sensor without any intermediate material.

The antibody (along with the conjugated chromic marker) may be conjugated to the polymer matrix such that the chromic marker is activated when the polymer matrix comes in contact with the biomarker of interest. The antibody may be conjugated to the polymer matrix using any conjugation technique known in the art. For example, the antibody may conjugated to the polymer matrix via biotin-avidin chemistry, succinimide chemistry, carbodiimide chemistry, isothicyanate chemistry, or any other suitable technique known in the art depending on the particular antibody, the chromic marker, the polymer and the biomarker or interest.

The antibody may be any antibody targeted to the biomarker of interest and the fluorescent marker or dye may be any suitable marker or dye that can conjugated to the antibody. The scope of the present disclosure is not limited by specific antibodies, but only by structure of the chromic sensor disclosed herein.

The chromic sensor disclosed herein may be incorporated into any suitable device that can be attached to the skin adjacent to the insertion site at which an IVC is inserted into the patient.

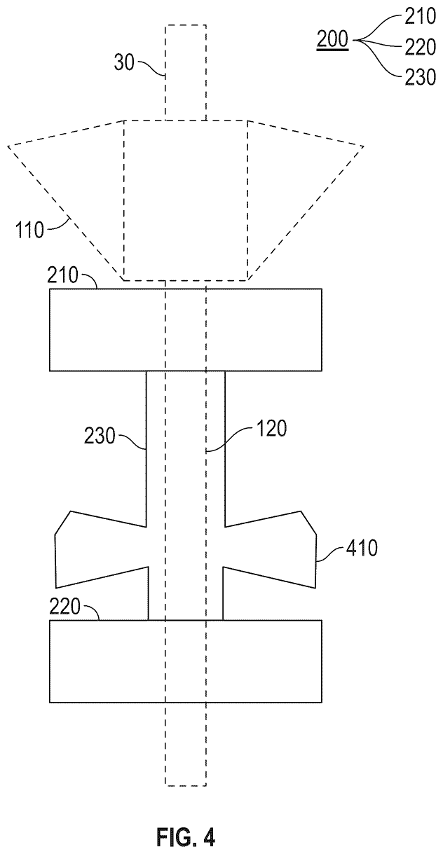

For example, FIG. 4 illustrates a schematic of a chromic sensor, e.g., chromic sensor 200, disclosed herein and a clip used for securing the chromic sensor to the skin of the patient. As seen in FIG. 4, the clip 410 secures the position of the first region 210 and the second region 220 of the chromic sensor 200. The clip 410 is designed and structured such that the first region 210 and the second region 220 of the chromic sensor are physically attached to the clip 410. In use, the clip 410 and the chromic sensor 200 are attached to the skin of the patient such that the first region 210 of the chromic sensor 200 is adjacent to the insertion site at which the tube 120 of the IVC is inserted into the patient, and the second region 220 of the chromic sensor 200 is distal to the insertion site.

In some implementations, IVC 100 may be designed such that the clip 410 is also physically attached to the hub 110 of the IVC 100. The clip 410 and the hub 110 may be detachably attached, or the attachment may be fixed. Thus, for example, the clip 410 may be attached to the hub 110 using a fastening mechanism such as a clip or a snap-fitting engagement member to enable detachment of the clip from the hub, in some implementations. In other implementations, the hub 110 and the clip 410 may be integrally formed or attached together using an adhesive so as to prevent detachment of the clip from the hub.

The attachment between the skin of the patient and the clip may be achieved via any suitable fastening mechanism. For example, a tape with a Velcro-like surface on one side and an adhesive on the other side may be adhered to the skin of the patient, and the clip 410 may have a corresponding Velcro-like surface enabling the clip 410 to attach to the skin of the patient via the tape. As another example, the clip 410 itself may have a skin-suitable adhesive enabling the clip 410 to attach directly to the skin of the patient.

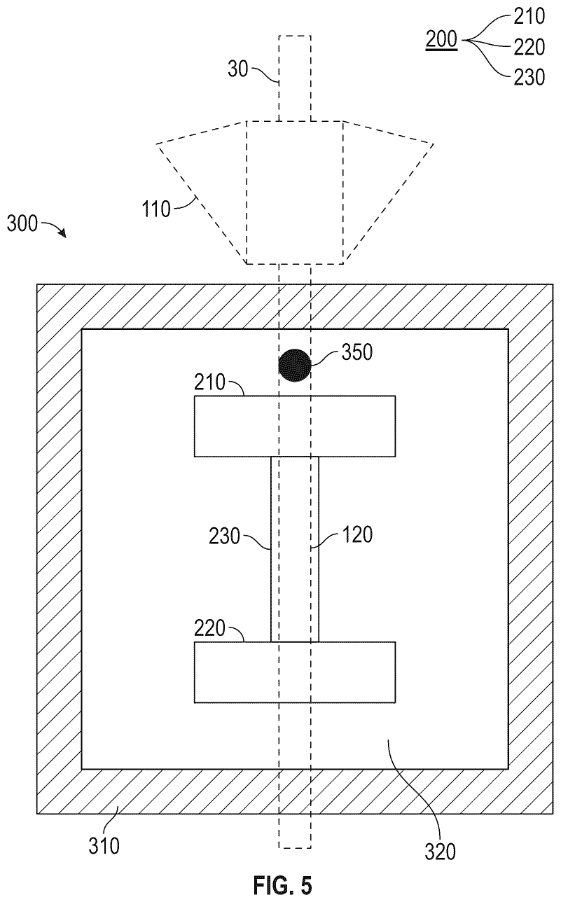

As another example, FIG. 5 illustrates a schematic of a dressing incorporating a chromic sensor, e.g., chromic sensor 200, disclosed herein. The dressing 300 includes a border portion 310, and a window portion 320, and has a skin-facing side and an opposing side. The dressing 300 is applied to the skin of the patient with the skin-facing side of the dressing facing and/or contacting the skin of the patient, at or around the insertion site through the an IVC, e.g., IVC 100, is inserted into the patient. The opposing side of the dressing faces away from the patient's skin.

In some implementations, a chromic sensor disclosed herein such as, for example, the chromic sensor 200, is disposed in the window portion 320, in particular, on the skin-facing side of the window portion 320. In some examples, the window portion 320 is made of a transparent or translucent material through which the skin of the patient around the insertion site is visible such that the any change in the color of the first and/or second regions of the chromic sensor is visible to a practitioner or care giver inspecting the insertion site. The border portion 310 includes an adhesive to enable the dressing 300 to adhere to the skin of the patient surrounding the insertion site.

In some examples, the dressing 300 may further include a marker 350 provided within the window portion 320 to be aligned with the catheter insertion site when applying the dressing 300 such that the first region 210 is proximal to the insertion site and the second region 220 is distal to the insertion site along a length of the inserted portion of the catheter. Thus, when the dressing 300 is applied to the skin of the patient to secure the catheter, the marker 350 enables the caregiver to suitably align the first and second regions of the chromic sensor with the catheter.

In use, a caregiver can periodically inspect the dressing 300, more particularly, the chromic sensor underlying the dressing 300 to monitor the state of the catheter to determine whether the color of one or both of the first and second regions has changed. Any change in conditions of the skin surround the insertion site, in particular, along the length of the inserted portion of the catheter can be detected by a change in color at the first and/or second regions of the chromic sensor underlying the dressing 300. For example, a change in temperature of the skin of the patient caused by, for example, a leak in the catheter because the temperature of the fluid being infused through the catheter is substantially lower than the skin temperature, may cause the temperature of the skin underlying the second region 220 to reduce more than that of the skin underlying the first region 210, thereby changing the color of the second region to be different from that of the first region.

Those of ordinary skill in the art, upon understanding the technology disclosed herein, would appreciate that the two regions of the chromic sensor serve multiple purposes. First, the two regions provide redundancy in measurement. Second, the two regions provide a mechanism for normalizing the measurement, in particular, when the measured quantity (e.g., temperature) is expected to affect regions of the skin underlying the two regions identically under normal conditions. Third, the two regions provide a differential measurement, e.g., when the quantity to be measured varies along the length of the inserted portion of the catheter.

Thus, depending on the particular quantity being measured, e.g., temperature, pH, concentration or presence of certain antigens, toxins or biomarkers, or mechanical strain, the two measurements obtained by the two regions of the chromic sensor can be beneficially used to monitor the area of the skin surrounding the insertion site to determine whether there are any complications or compromises surrounding the insertion site.

In some implementations, a chromic sensor and a dressing provided separately such that the caregiver may apply the chromic sensor to the skin of the patient first and then secure the chromic sensor and the catheter together with the dressing. In some implementations, chromic sensor may be pre-attached to the dressing such that the chromic sensor is automatically attached to the skin of the patient when the caregiver secures the catheter using the dressing.

In some implementations, the chromic sensor may be included as part of a kit comprising an intravenous catheter, the chromic sensor and a fastener (e.g., a dressing disclosed herein or another fastener) designed and constructed to enable the chromic sensor to be attached to the skin surrounding the insertion site at which the catheter is inserted into the patient. The fastener may include an adhesive, a clip or any other suitable device that can attach the chromic sensor to the skin and/or the hub of the catheter adjacent the insertion site. For instance, the fastener may be a transparent or translucent adhesive that can be applied over the chromic sensor and the skin surrounding the insertion site, so as to allow visual monitoring of the chromic sensor. In some implementations, the kit may further include instructions about using the chromic sensor and attaching the chromic sensor to the skin of the patient. In some implementations, the instructions may include a color guide to enable a caregiver to correlate a condition with a color of one or both of the regions of the chromic sensor and make a quick diagnosis of the condition indicated by the response of the chromic sensor. In some implementations, the color guide may be included in the kit. Additionally or alternatively, the color guide may be printed on or attached to the fastener used for attaching the chromic sensor to the skin of the patient, facilitating the caregiver to visually monitor the insertion site and the surround skin area.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1: A chromic sensor for an intravenous catheter, comprising a first region disposed to be proximal to an insertion site at which the intravenous catheter is inserted into a patient, the first region including a chromic material that changes color in response to a stimulus; and a second region disposed to be distal to the insertion site, spaced apart from the first region along a portion of the intravenous catheter inserted into the patient, the second region including the chromic material. The first and second regions are configured to respond identically to identical stimuli.

The chromic sensor of clause 1, wherein the stimulus is selected from the group consisting of: change in temperature, change in pH, mechanical stress or strain, presence of a chemical entity, and a combination thereof.

The chromic sensor of clause 1, wherein the chromic material comprises a thermochromic liquid crystal or a leuco dye.

The chromic sensor of clause 3, wherein the chromic material changes color in response to a temperature change ranging from about 0.1° C. to about 10° C..

The chromic sensor of clause 1, wherein the chromic material comprises a sulfophthalein dye, a phthalein dye, an azo dye, a 1, 8-naphthalimide dye, a boron-dipyrromethene dye, a diketopyrrolopyrrole dye, a cyanine dye, or a fluorescein.

The chromic sensor of clause 1, wherein the chromic material changes color in response to a change in pH ranging from about 1.0 to about 3.0.

The chromic sensor of clause 1, wherein the chromic material changes color in response to mechanical strain ranging from about 0.5% to about 3.0%.

The chromic sensor of clause 7, wherein the chromic material comprises mechanochromic dyes selected from the group consisting of: spiropyrans, spirooxazines, diarylethenes, metal-organic frameworks, liquid crystal polymers and a combination thereof.

The chromic sensor of clause 1, wherein the chromic material comprises microcapsules having a solvent reactive dye which changes color in response to contact with extracellular fluids.

The chromic sensor of clause 1, wherein the chromic material comprises a polymer matrix conjugated with an antibody tethered to a fluorescent marker.

The chromic sensor of clause 10, wherein the antibody comprises an immunoglobulin.

The chromic sensor of clause 10, wherein the polymer matrix comprises a polymer selected from the group consisting of: polyacrylic acid, hyaluronic acid, polyethylene glycol, and a combination thereof.

The chromic sensor of any of clauses 1-12, further comprising a fastener configured to attach the chromic sensor to the intravenous catheter.

The chromic sensor of clause 13, wherein the fastener is disposed between the first and second regions.

A dressing comprising a chromic sensor of any of clauses 1-14.

The dressing of clause 15, further comprising a transparent or translucent material to enable visualization of a portion of the patient's skin at and surrounding the insertion site through the dressing.

The dressing of clause 16, further comprising a visual marker positioned to align with the insertion site.

An intravenous catheter comprising the chromic sensor of any of clauses 1-14.

A kit comprising: an intravenous catheter and a chromic sensor. The chromic sensor comprises: a first region disposed to be proximal to an insertion site at which the intravenous catheter is inserted into a patient, the first region including a chromic material that changes color in response to a stimulus, and a second region disposed to be distal to the insertion site, spaced apart from the first region along a portion of the intravenous catheter inserted into the patient, the second region including the chromic material. The first and second regions are configured to respond identically to identical stimuli. The kit further comprises a fastener configured to attach the chromic sensor to the intravenous catheter adjacent the insertion site at which the catheter is inserted into the patient.

A method of visually monitoring whether an intravenous catheter, having the chromic sensor of any of clauses 1-14 provided therewith, inserted into a patient and/or an insertion site at which the intravenous catheter is inserted into the patient is compromised, the method comprising: visually determining a color of each of the first region and the second region of the chromic sensor; and determining that the catheter and/or the insertion site is compromised if the color of the first region is different from the color of the second region.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

In one or more aspects, the terms “about,” “substantially,” and “approximately” may provide an industry-accepted tolerance for their corresponding terms and/or relativity between items, such as from less than one percent to five percent.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A chromic sensor for an intravenous catheter, comprising:
a first region disposed to be proximal to an insertion site at which the intravenous catheter is inserted into a patient, the first region including a chromic material that changes color in response to a stimulus; and
a second region disposed to be distal to the insertion site, spaced apart from the first region along a portion of the intravenous catheter inserted into the patient, the second region including the chromic material,
wherein the first and second regions are configured to respond identically to identical stimuli.

2. The chromic sensor of claim 1, wherein the stimulus is selected from the group consisting of: change in temperature, change in pH, mechanical stress or strain, presence of a chemical entity, and a combination thereof.

3. The chromic sensor of claim 1, wherein the chromic material comprises a thermochromic liquid crystal or a leuco dye.

4. The chromic sensor of claim 3, wherein the chromic material changes color in response to a temperature change ranging from about 0.1° C. to about 10° C..

5. The chromic sensor of claim 1, wherein the chromic material comprises a sulfophthalein dye, a phthalein dye, an azo dye, a 1, 8-naphthalimide dye, a boron-dipyrromethene dye, a diketopyrrolopyrrole dye, a cyanine dye, or a fluorescein.

6. The chromic sensor of claim 1, wherein the chromic material changes color in response to a change in pH ranging from about 1.0 to about 3.0.

7. The chromic sensor of claim 1, wherein the chromic material changes color in response to mechanical strain ranging from about 0.5% to about 3.0%.

8. The chromic sensor of claim 7, wherein the chromic material comprises mechanochromic dyes selected from the group consisting of: spiropyrans, spirooxazines, diarylethenes, metal-organic frameworks, liquid crystal polymers and a combination thereof.

9. The chromic sensor of claim 1, wherein the chromic material comprises microcapsules having a solvent reactive dye which changes color in response to contact with extracellular fluids.

10. The chromic sensor of claim 1, wherein the chromic material comprises a polymer matrix conjugated with an antibody tethered to a fluorescent marker.

11. The chromic sensor of claim 10, wherein the antibody comprises an immunoglobulin.

12. The chromic sensor of claim 10, wherein the polymer matrix comprises a polymer selected from the group consisting of: polyacrylic acid, hyaluronic acid, polyethylene glycol, and a combination thereof.

13. The chromic sensor of claim 1, further comprising a fastener configured to attach the chromic sensor to the intravenous catheter.

14. The chromic sensor of claim 13, wherein the fastener is disposed between the first and second regions.

15. A dressing comprising a chromic sensor of claim 1.

16. The dressing of claim 15, further comprising a transparent or translucent material to enable visualization of a portion of the patient's skin at and surrounding the insertion site through the dressing.

17. The dressing of claim 16, further comprising a visual marker positioned to align with the insertion site.

18. An intravenous catheter comprising the chromic sensor of claim 1.

19. A kit comprising:
an intravenous catheter;
a chromic sensor comprising:
a first region disposed to be proximal to an insertion site at which the intravenous catheter is inserted into a patient, the first region including a chromic material that changes color in response to a stimulus, and
a second region disposed to be distal to the insertion site, spaced apart from the first region along a portion of the intravenous catheter inserted into the patient, the second region including the chromic material,
wherein the first and second regions are configured to respond identically to identical stimuli; and
a fastener configured to attach the chromic sensor to the intravenous catheter adjacent the insertion site at which the catheter is inserted into the patient.

20. A method of visually monitoring whether an intravenous catheter, having the chromic sensor of claim 1 provided therewith, inserted into a patient and/or an insertion site at which the intravenous catheter is inserted into the patient is compromised, the method comprising:
visually determining a color of each of the first region and the second region of the chromic sensor; and
determining that the catheter and/or the insertion site is compromised if the color of the first region is different from the color of the second region.
